(19) United States
(12) Patent Application Publication
(10) Pub. No.: US 2013/0131544 A1
(43) Pub. Date: May 23, 2013

(54) BIOPSY FORCEPS INCLUDING SELF ALIGNING JAWS WITH DRAINAGE OPENINGS

(71) Applicant: Anrei Medical (HZ) Co., Ltd., Hangzhou (CN)

(72) Inventors: Mark A. Bowden, West Warwick, RI (US); Raymond A. Frechette, North Providence, RI (US); Chris DeCarton, Wrentham, MA (US); Changqing Li, Bloomington, IN (US); Matthew Otten, Cincinnati, OH (US)

(73) Assignee: ANREI MEDICAL (HZ) CO., LTD., Hangzhou (CN)

(21) Appl. No.: 13/672,007

(22) Filed: Nov. 8, 2012

Related U.S. Application Data
(60) Provisional application No. 61/561,035, filed on Nov. 17, 2011.

(51) Int. Cl. A61B 10/06; A61B 10/02

(52) U.S. Cl. CPC A61B 10/06; A61B 10/0266
USPC 600/564

(57) ABSTRACT

Improved biopsy forceps include pivotally coupled self aligning jaws with drainage holes therethrough. The biopsy forceps include a pair of jaw members that include cup shaped jaws. The biopsy forces may be formed by a series of metal etching and forming operations to allow the manufacture of the biopsy forceps using high volume manufacturing techniques.

### CROSS REFERENCE TO RELATED APPLICATIONS

The present application claims priority benefit of U.S. Provisional Patent Application No. 61/561,035 filed Nov. 17, 2011 and titled BIOPSY FORCEPS INCLUDING JAWS WITH DRAINAGE OPENINGS.

### STATEMENT REGARDING FEDERALLY SPONSORED RESEARCH OR DEVELOPMENT

Not applicable

### FIELD OF THE INVENTION

The present invention relates generally to biopsy forceps and, more specifically, to biopsy forceps having self aligning jaws and drainage openings through the jaws.

### BACKGROUND OF THE INVENTION

Biopsies are a common medical procedure in which a tissue sample is taken from a patient for further analysis using biopsy forceps. Various designs of biopsy forceps are known for this purpose. Typical biopsy forceps include a pair of jaws that can be opened and closed manually by a physician to excise a small sample of tissue. Biopsy forceps designs are often complex and costly to produce due to the need to provide a precision medical device with precisely aligned jaws. Biopsy forceps are often considered single use devices, i.e. they are disposed of following a single use. Given the large number of biopsies that are performed and given that they are typically single use devices, it is desirable to be able to produce these devices at high volumes and at low cost. The complexity of biopsy forceps design involves the need to provide a precision medical device at low cost.

Additionally, when tissue samples are obtained, they often include substantial liquid content with the tissue sample. The liquid content reduces the size of the tissue sample that that be obtained for a given jaw size.

It would therefore be desirable to have biopsy forceps that could be produced using high volume manufacturing techniques that avoided the disadvantages of known biopsy forceps.

### BRIEF SUMMARY OF THE INVENTION

Improved biopsy forceps are disclosed that have self aligning jaws and drainage openings. The biopsy forces includes a jaw assembly having a pair of pivotally interconnected jaw members. Each jaw member has a cup shaped jaw at one end. When pivotally interconnected, the jaws are in opposed relation and can be manually opened and closed. The jaws have a peripheral edge that is sharpened or that includes teeth to facilitate the cutting of tissue upon closure of the opposed jaws.

One embodiment of the jaws includes beveled offset self aligning teeth along the peripheral edge of the jaw that provide for clean cutting of tissue. Another embodiment includes teeth that are generally pyramidally shaped with beveled surfaces. Alternatively, a single sharpened peripheral edge on either side of the jaw cooperates with a single sharpened edge on either side of the opposed jaw to provide cutting edges to sever tissue upon closure of the opposed jaws. The teeth in opposed jaws are configured and cooperative to provide self-alignment of the jaws upon closure thereof.

The jaws include at least one fenestration hole or opening through the jaw to allow for the drainage of liquid therethrough. The openings through the opposed jaws allow for the capture of a greater amount of tissue for the same jaw size and additionally, allow for captured tissue to be more easily rinsed off in view of the improved drainage from the jaws.

Other features, aspects and advantages of the invention will be evident from the Drawings and the Detailed Description of the Invention that follow.

### BRIEF DESCRIPTION OF THE SEVERAL VIEWS OF THE DRAWINGS

The foregoing and other objects, features and advantages of the invention will be apparent from the Detailed Description of the Invention in conjunction with the drawings of which:

FIG. 1a is an illustration of one embodiment of a jaw member for use in biopsy forceps in which the jaw includes generally pyramidally shaped self aligning teeth and fenestration holes for improved drainage in accordance with the present invention;

FIG. 1b illustrates a blank used in the fabrication of the jaw member of FIG. 1a following etching of the blank;

FIG. 2a is another embodiment of a jaw member including offset beveled self aligning teeth and fenestration holes for use in the fabrication of biopsy forceps in accordance with the present invention;

FIG. 2b is a blank used in the fabrication of a jaw member having offset beveled self-aligning teeth and fenestration holes through the jaw for use in the fabrication of biopsy forceps in accordance with the present invention; and

FIGS. 3a and 3b depict biopsy forceps with alternative actuation members for opening and closing the jaws of biopsy forceps such as illustrated in FIGS. 1a and 2a.

### DETAILED DESCRIPTION OF THE INVENTION

The disclosure of U.S. Provisional patent application No. 61/561,035 filed Nov. 17, 2011 and titled Biopsy Forceps Including Jaws with Drainage Openings is hereby incorporated by reference in its entirety.

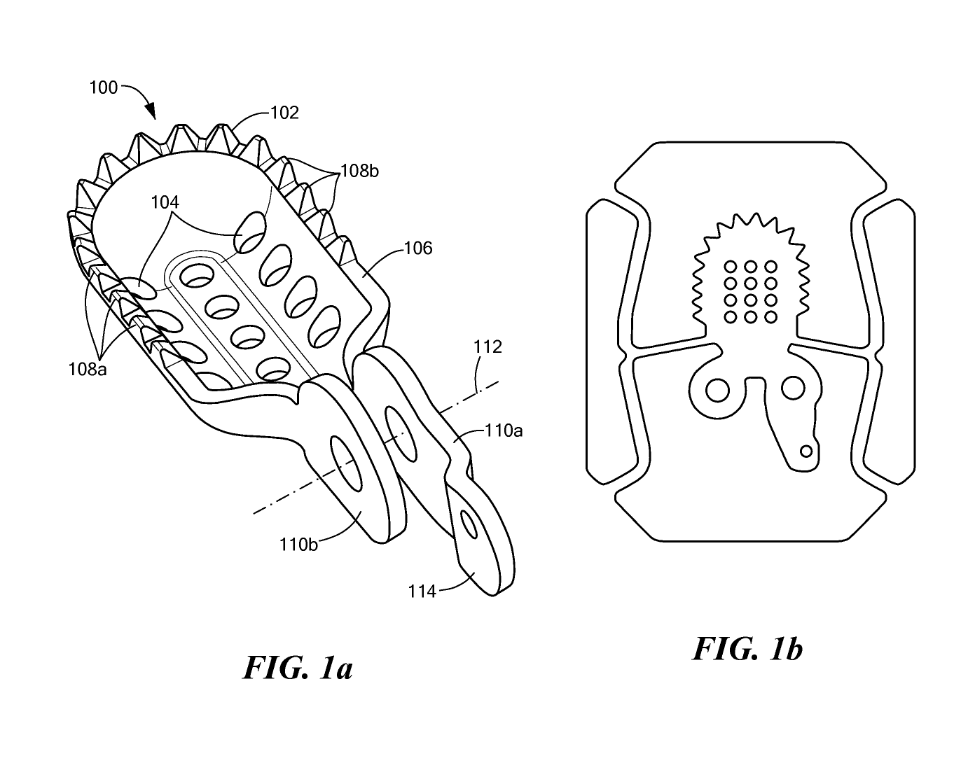

FIG. 1a illustrates a jaw member 100 that is employed in one embodiment of biopsy forceps in accordance with the present invention. The jaw member 100 includes a concave cup shaped jaw portion 102 that includes an array of openings or fenestration holes 104 to allow for the drainage of liquid therethrough upon acquisition of a tissue specimen. Additionally, the drainage holes allow for more thorough rinsing of the tissue sample. The cup shaped jaw 102 has a peripheral edge 106 having teeth 108a, 108b extending therefrom. The jaw members 100 and components of the biopsy forceps are fabricated of stainless steel or any other suitable medical grade material. The teeth 108a on one side of the cup shaped portion 102 are configured and offset from the teeth 108b on the opposite side of the cup shaped portion 102 so that the teeth of opposed jaws mesh when the opposing jaws 102 are closed. In the embodiment illustrated in FIG. 1a, the teeth 108a and 108b are generally pyramidally shaped and are arranged on the peripheral edge 106 of the jaw 102 such that the teeth 108a of one jaw member 100 mesh with the teeth 108b of an opposed jaw member 100 when two jaw members 100 are pivotally coupled as subsequently discussed. The generally pyramidally shaped teeth have surfaces that engage surfaces of offset generally pyramidally shaped teeth of the opposing jaw to self align the jaws upon closure. Two of the jaw members 100 having the same shape, may be pivotally coupled to form the biopsy forceps with the teeth of each jaw 102 disposed between teeth of the opposing jaw when the jaws 102 of the opposed jaw members 100 are closed. The teeth on one side of the jaw are offset from the teeth on the opposite side of the same jaw to allow the teeth to mesh when two of the jaw members of the same shape are pivotally mated. While it is noted that it is desirable for the jaw members to have the same shape to facilitate high volume and low cost manufacture of the same, it should be recognized that the tooth patterns may be specified so that the jaws achieve self alignment but opposing jaw members do not have the same configuration. In this circumstance, two different jaw members would need to be manufactured.

The cup shaped jaws 102 include a distal end at the frontal or leading edge of the jaw and a proximal end at the rearward end of the cup shaped jaw 102. A pair of opposed flanges 110a, 110b extend from the proximal end of the cup shaped jaw 102. The flanges 110a, 110b include opposed coaxially aligned openings defining a pivot axis 112. Two of the jaws members 100 are pivotally coupled via a pivot pin 220 that extends through coaxially aligned openings in the flanges 110a, 110b of the opposed jaw members 100.

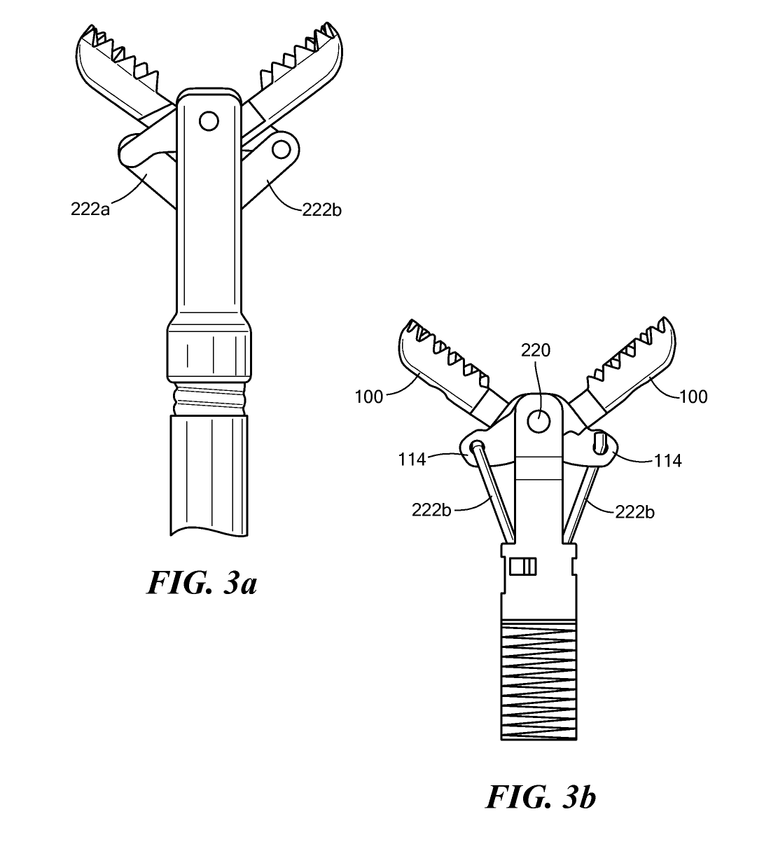

An actuation arm 114 extends from the flange 110a of each jaw member 100 and is formed integrally and as a single piece therewith. When the jaw members 100 are pivotally coupled, actuation members 222a, 222b are coupled to each actuation arm 114 as depicted in FIGS. 3a and 3b, respectively. Movement of the actuation arm 114 away from the pivot axis 112 via the actuation members (generally downward as illustrated in FIGS. 3a and 3b) causes the jaws 102 of the biopsy forceps to close and movement of the actuation arm 114 toward the pivot axis 112 (generally upward in FIGS. 3a and 3b as shown) causes the jaws of the biopsy forceps to open.

The jaws members 100 of the presently disclosed biopsy forceps are fabricated by first etching a generally planar blank and then forming the jaw 102 into the configuration illustrated in FIG. 1a. The etching process includes etching of the planar blank to form the teeth 108a, 108b as well as the array of fenestration holes 104.

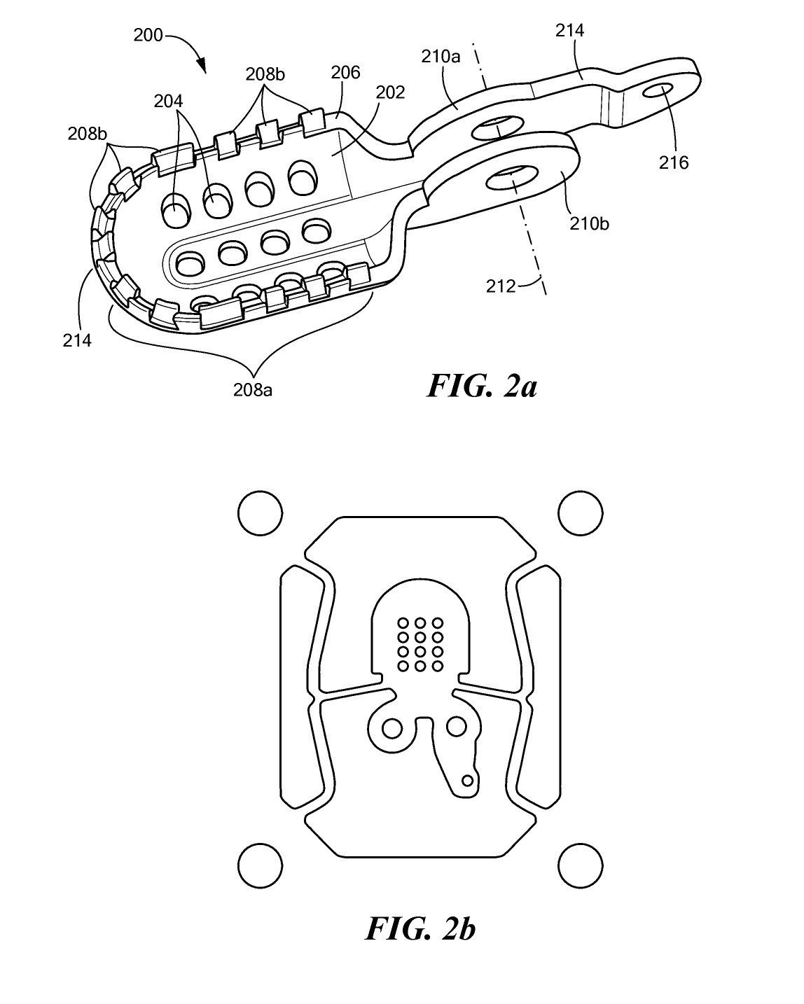

Another embodiment of a jaw member 200 for use in biopsy forceps in accordance with the present invention is illustrated in FIG. 2a. The jaw member 200 and components of the biopsy forceps are fabricated of stainless steel or any other suitable medical grade material. The jaw member 200 includes a cup shaped jaw 202 having an array of fenestration holes 204 formed therethrough to allow for drainage of liquid. The cup shaped jaw 202 includes a peripheral edge 206 having a plurality of teeth 208a, 208b extending therefrom. The teeth 208a on one side of the jaw are beveled and offset laterally from the teeth 208b on the opposing side of the same jaw. A pair of jaw members 200 of the type illustrated in FIG. 2a are employed to produce the biopsy forceps. More specifically, at least some of the teeth along the peripheral edge 206 of each of the jaws 202 include a tooth surface that is slanted outwardly away from the cup shaped jaw of the respective jaw and at least some of the teeth along the peripheral edge of each of the jaws 202 include a tooth surface that is slanted inwardly toward the cup shaped portion of the respective jaw 202. Outwardly and inwardly slanted tooth surfaces of opposed jaws are disposed in opposed relation and cooperative to provide self alignment of the jaws upon closure of the opposed jaws and engagement of the opposed inwardly and outwardly slanted tooth surfaces.

The jaw members 200 include flanges 210a, 210b having openings therethrough defining a pivot axis 212. Flanges 210a include an actuation arm 214 integrally formed therewith as a single piece for attachment of an actuating member 222a, 222b. The actuating arm 214 of each jaw member 200 includes an opening 216 for coupling the actuating member thereto. As illustrated in FIGS. 3a and 3b, actuating members 222a, 222b, may be in the form of pivotally coupled flanges or rod-like linkages.

When a pair of jaw members 200 are pivotably coupled with the teeth of the respective jaws 202 in opposed relation, as a result of the beveling of teeth on opposed jaws and the positioning of the teeth along the peripheral edge of the jaws, upon closure of the jaws, the teeth serve to provide for self alignment of the jaws 202 with respect to one another. While plural teeth are shown on either side of a center line 215 bisecting a jaw 202, is should be appreciated that a single tooth or knife edge may be provided on each side of the centerline 215 along the peripheral edge 206. When a pair of jaw members 200 are pivotally coupled, the teeth or tooth 208a of one jaw 202 oppose the teeth or tooth 208b of the other jaw 202 so that upon closure of the jaws, the tissue is cleanly severed.

The actuating members 222a, 222b are pivotally coupled to the actuating arms 114, 214 as illustrated in FIG. 3a or 3b to provide for opening and closure of the jaws 102, 202 respectively.

As discussed in conjunction with FIG. 1b, the jaw member 200 is formed by first etching a blank, such as illustrated in FIG. 2b and then forming the etched blank to obtain the cup shaped jaw member 200.

In the above-discussed examples, the jaw members 100, 200 each include a pair of opposed flanges. It should be recognized that each jaw members may be fabricated with either one or two flanges. The provision of two flanges should provide more consistent alignment of the jaws since the pivot pin extends through two coaxially aligned openings in each jaw member when two flanges per jaw member are provided.

It should also be appreciated that variations of and modifications to the above-described biopsy forceps may be made without departing from the inventive concepts disclosed herein.

What is claimed is:

1. Biopsy forceps comprising:
a biopsy jaw assembly including first and second jaw members, each of the jaw members having a proximal end and a distal end, the jaw members each being formed as an integral single piece member and including:
a concave cup shaped jaw having a peripheral edge, a forward end corresponding to the distal end of the respective jaw member and a rearward end;
a flange extending from the rearward end of the cup shaped jaw, the flange including an opening defining a pivot axis, wherein the rearward end of the cup shaped jaw is proximal to the pivot axis; and
an actuating arm extending from the flange and terminating at the proximal end of the respective jaw member, the actuating arm configured for coupling to an actuating member; and
the first and second jaws disposed in opposing relation with the openings in flanges of the first and second jaw members coaxially aligned;
a pivot pin pivotally extending through the coaxially aligned flange openings and coupling the first and second jaw members to permit pivotal rotation of the cup shaped jaws about the pivot axis and opening and closure of the cup shaped jaws in response to movement of the actuating arms of the jaw shaped members,
wherein the cup shaped jaw of each jaw member includes a plurality of teeth configured and positioned so as to cooperate with the plurality of teeth of the opposing jaw to provide self-alignment of the first and second jaws upon closure thereof.

2. The biopsy forceps of claim 1 wherein each jaw includes a plurality of teeth along the peripheral edge of the jaw on either side of a centerline bisecting the jaw wherein the plurality of teeth on each side of the centerline of each jaw are cooperative with a plurality of teeth on the opposing jaw to provide self alignment of the opposing jaws upon closure thereof.

3. The biopsy forceps of claim 1 wherein at least some of the teeth along the peripheral edge of each of the jaws include a tooth surface that is slanted outwardly away from the cup shaped portion of the respective jaw and at least some of the teeth along the peripheral edge of each of the jaws include a tooth surface that is slanted inwardly toward the cup shaped portion of the respective jaw and the outwardly and inwardly slanted tooth surfaces of opposed jaws are disposed in opposed relation and cooperative to provide self alignment of the jaws upon closure of the opposed jaws and engagement of the opposed inwardly and outwardly slanted tooth surfaces.

4. The biopsy forceps of claim 1 wherein at least some of the plurality of teeth have a generally pyramidal shape and are disposed along the peripheral edge of each jaw such that the generally pyramidal teeth of one of the jaws are positioned between the generally pyramidal teeth of the opposing jaw with tooth surfaces of one of the jaws abutting tooth surfaces of the opposing jaw to provide self alignment of the jaws upon closure of the jaws.

5. The biopsy forceps of claim 1 further including first and second actuating members pivotably coupled to respective actuating arms of the first and second jaws, the actuating members operative to open and close the opposed jaws via pivotal movement of the jaws about the pivot pin in response to movement of the actuating members generally toward and away from the jaws respectively.

6. The biopsy forceps of claim 1 where the first and second jaw members have the same shape.

7. The biopsy forceps of claim 1 including at least one opening through the at least one of the cup shaped jaws to permit the drainage of liquid therethrough.

8. The biopsy forceps of claim 1 wherein each of the cup shaped jaws includes a plurality of openings to permit the drainage of liquid therethrough.

9. The biopsy forceps of claim 1 wherein each jaw member includes a pair of opposed flanges extending from the rearward end of the cup shaped jaw, the pair of opposed flanges including coaxially aligned openings defining the pivot axis,
wherein the actuating arm extends from one flange of the pair of flanges of each jaw member and the pivot pin extends through the coaxially aligned openings of the pairs of flanges; and
the first and second jaw assemblies being disposed with pivot axes through the flanges aligned and with jaws of the first and second jaw members in opposed relation.

10. The biopsy forceps of claim 1 further including;
a biopsy jaw member support having a jaw member supporting end and a longitudinal opening extending through the jaw member support, the jaw member support having coaxially aligned pivot openings at the supporting end generally transverse to a longitudinal axis through the longitudinal opening, wherein the pivot pin extends through the pivot openings of the jaw member support to mount the jaw members to the jaw member support;
actuating members extending through the jaw member support and coupled to the actuating arms to provide opening and closure of the jaws of the biopsy forceps upon longitudinal movement of the actuating members within the jaw member support in first and second opposing directions.

11. Biopsy forceps comprising:
a biopsy jaw assembly including first and second jaw members, each jaw member including a concave cup shaped jaw, wherein the jaws of each jaw member are disposed in opposed relation and movable between an open and a closed position,
wherein at least one of the concave cup shaped jaws has at least one opening through the cup shaped jaw portion to allow for the drainage of liquid therethrough.

12. The biopsy forceps of claim 11 wherein each of the jaw members has a proximal end and a distal end, the concave cup shaped jaw has a peripheral edge, a forward end and a rearward end, the jaw members each being formed as an integral single piece member and including:
the concave cup shaped jaw;
at least one flange extending from the rearward end of the cup shaped portion, the flange including an opening sized to receive a pivot pin;
an actuating arm extending from the at least one flange and terminating at the proximal end of the respective jaw member, the actuating arm configured for coupling to an actuating member; and
the first and second jaws disposed in opposing relation with the openings in each of the at least one flange of the first and second jaw members coaxially aligned to define a pivot axis;
a pivot pin pivotally extending through the coaxially aligned openings of the at least one flange of the first and second jaw members to permit pivotal rotation of the cup shaped jaws about the pivot axis and opening and closure of the cup shaped jaws in response to movement of the actuating arms of the first and second jaw members.

13. The biopsy forceps of claim 11 further including;
a biopsy jaw member support having a jaw member supporting end and a longitudinal opening extending through the jaw member support, the jaw member support having coaxially aligned pivot openings at the jaw member supporting end, wherein the pivot pin extends through the pivot openings of the jaw member support to mount the jaw members to the jaw member support;
actuating members extending through the jaw member support and coupled to the actuating arms to provide opening and closure of the jaws of the biopsy forceps upon longitudinal movement of the actuating members within the jaw member support in first and second generally opposing directions.
